(19) United States
(12) Patent Application Publication
(10) Pub. No.: US 2024/0391788 A1
(43) Pub. Date: Nov. 28, 2024

(54) PREPARATION OF ALKALI SALT SOLUTIONS

(71) Applicant: Li-Technology Pty Ltd, West Perth (AU)

(72) Inventors: Gary Donald JOHNSON, Subiaco (AU); John Wing Keung LAU, Villanova, PA (US)

(73) Assignee: Li-Technology Pty Ltd, West Perth (AU)

(21) Appl. No.: 18/696,609

(22) PCT Filed: Sep. 27, 2022
(86) PCT No.: PCT/AU2022/051154

§ 371 Date: Mar. 28, 2024

(30) Foreign Application Priority Data
Oct. 1, 2021 (AU) 2021903148

(51) Int. Cl. C01D 17/00; B01J 23/22; C05D 1/00

(52) U.S. Cl. CPC C01D 17/00; B01J 23/22; C05D 1/00

(57) ABSTRACT

A process of preparing an alkali salt solution for the production of catalysts or promoters of catalysts, the process comprising the steps of: (i) Passing a solution containing a mixture of potassium, rubidium and caesium sulfates through an evaporative crystallisation step to obtain a potassium containing salt, and a solution comprising rubidium and caesium sulfate; (ii) Further evaporative crystallisation of the liquid product of step (i) to obtain a rubidium containing salt and a solution enriched in caesium sulfate; and (iii) Mixing one or more of the obtained salts and the solution at a desired ratio to prepare the alkali salt solution.

### FIELD OF THE INVENTION

The present invention relates to a process of preparing alkali salt solutions.

More particularly, the process of the present invention is intended for use in the preparation of alkali salt solutions suitable for use as catalysts or promoters of catalysts.

In one form, the process of preparing the alkali salt solutions of the present invention utilises an intermediate product of a process for the recovery of lithium. Said intermediate product is capable of producing different ratios of potassium, rubidium and caesium suitable for use as catalysts or promoters of catalysts.

More particularly, the process of the present invention is intended to produce promoters of vanadium pentoxide catalysts for the oxidisation of sulfur dioxide to sulfur trioxide. Still further, the process of the present invention is intended to produce catalysts for the aldol condensation of formaldehyde with alkyl esters or acids to produce ethylenically unsaturated esters or acids.

### BACKGROUND ART

Vanadium pentoxide catalysts are typically used to catalyse the oxidisation of sulfur dioxide to sulfur trioxide in the manufacture of sulfuric acid:

$$2 {SO}_{2 ( g )} + {O}_{2 ( g )} ↔ 2 {SO}_{3 ( g )}$$

With vanadium catalysts, an alkali promotor, typically potassium, in the form of potassium sulfate is used to accelerate the oxidisation reaction.

The mixture of potassium sulfate and vanadium pentoxide on the surface of a silica-based support melts at a specific temperature and it is at or within this molten surface that the catalytic reactions occur.

More recently, caesium, typically in the form of caesium sulfate, has replaced at least some potassium sulfate in the mixture with vanadium pentoxide. Specifically, caesium lowers the melting point of the mixture, allowing the catalysed reactions to occur at a lower temperature. Operating at a lower temperature is beneficial in the convertor of the acid plant. In particular, when used in the first bed of the convertor, operating at a lower temperature allows a faster ramp up or reboot of the acid plant after a shut down. When used in the last bed of the convertor, higher overall SO₂ to SO₃ conversion is achieved leading to a reduction in SO₂ concentrate in the tail gas.

Presently, the majority of caesium used in vanadium pentoxide catalysts are prepared by methods such as refining of ore containing the mineral pollucite ((Cs, Na)₂Al₂Si₄O₁₂·H₂O) which contains about 28% of caesium in its pure form. Importantly, there are few commercial deposits of pollucite remaining internationally.

Rubidium, being another alkali metal that has a higher atomic weight than potassium but lower than caesium, is also recognised as an effective promotor for vanadium catalysts as it has similar properties to caesium. Rubidium is generally not present at high concentrations in known minerals, other than lepidolite. Rubidium, in the form of rubidium oxide, can be present in lepidolite at up to 3.5% wt/wt. As such, it has not generally been used in commercial applications due to a lack of supply.

International Patent Application PCT/AU2015/000608 (WO 2016/054683) by the present Applicant describes a novel process for the recovery of lithium from lithium bearing mica rich minerals such as lepidolite, a naturally occurring mineral that contains significant amount of lithium whilst also comprising potassium, rubidium and caesium. The process described in Application PCT/AU2015/000608 utilises a combination of several process steps to selectively recover valuable metals, primarily recovering lithium as a lithium containing salt.

Application PCT/AU2015/000608 further describes the production of an alkali rich monovalent alum removed from the filtrate containing the majority of lithium from the lepidolite ore or concentrate. After precipitating the aluminium from the alum, a liquor containing potassium, caesium and rubidium in the form of a sulfate solution is obtained. Said liquor is subject to a selective crystallisation step, recovering individual salts of each alkali metal. Recovering individual salts of each alkali metal involve high costs due to the energy intensive process to crystallise each alkali metal salt.

The Applicant has identified that such an intermediate, being the liquor containing potassium, caesium and rubidium in the form of a sulfate solution, may be a potential feedstock in preparing an alkali salt solution for manufacture of catalysts or promoters of catalysts.

The method of the present invention has as one object thereof to overcome substantially the abovementioned problems of the prior art, or to at least provide a useful alternative thereto.

The preceding discussion of the background art is intended to facilitate an understanding of the present invention only. This discussion is not an acknowledgement or admission that any of the material referred to is or was part of the common general knowledge as at the priority date of the application.

Throughout the specification, unless the context requires otherwise, the word “comprise” or variations such as “comprises” or “comprising”, will be understood to imply the inclusion of a stated integer or group of integers but not the exclusion of any other integer or group of integers.

Each document, reference, patent application or patent cited in this text is expressly incorporated herein in their entirety by reference, which means that it should be read and considered by the reader as part of this text. That the document, reference, patent application, or patent cited in this text is not repeated in this text is merely for reasons of brevity.

Reference to cited material or information contained in the text should not be understood as a concession that the material or information was part of the common general knowledge or was known in Australia or any other country.

### DISCLOSURE OF THE INVENTION

In accordance with the present invention there is provided a process of preparing an alkali salt solution for the production of catalysts or promoters of catalysts, the process comprising the steps of:
(i) Passing a solution containing a mixture of potassium, rubidium and caesium sulfates through an evaporative crystallisation step to obtain a potassium rich salt, and a liquid product comprising rubidium and caesium sulfate;
(ii) Further evaporative crystallisation of the liquid product of step (i) to obtain a rubidium rich salt and a liquid product enriched in caesium sulfate; and
(iii) Mixing one or more of the obtained salts and the liquid product at a desired ratio to prepare the alkali salt solution.

Preferably, the potassium, rubidium and caesium containing solution is a co-product of a process for the recovery of lithium from lepidolite.

Preferably, the potassium containing salt is potassium sulfate.

Preferably, the rubidium containing salt is rubidium sulfate.

Still preferably, the rubidium sulfate may be further treated by way of established techniques to yield rubidium carbonate or rubidium hydroxide.

Preferably, the caesium containing solution is caesium sulfate, which can be further treated by way of established techniques to yield caesium carbonate or caesium hydroxide.

It is understood that each of the enriched sulfate solutions produced will contain minor amounts of each of the other alkali metals contained in the solution of step (i) or alum.

Preferably, the alkali salt solution is a promoter in the manufacture of a catalyst.

Still preferably, the catalyst is a vanadium pentoxide (V₂O₅) catalyst.

Yet still preferably, the vanadium pentoxide catalyst is used during oxidisation of sulfur dioxide (SO₂) to sulfur trioxide (SO₃).

In one form of the present invention the process provides a potassium sulfate product utilised in fertilisers.

The composition of the potassium, rubidium and caesium containing sulfate can all be represented by the generic formula M₂SO₄, where M represents the alkali metals present and is equal to A+B+C where A=the molar concentration of potassium, B=the molar concentration of rubidium and C=the molar concentration of caesium.

By mixing portions of the potassium containing salt, the rubidium containing salt and the caesium enriched solution it is possible to create a wide range of compositions for the resulting mixture. Where M=1 the concentration of each of A, B and C can be varied from about 0.1 to 1.0 provided the sum must add to 1.

The alkali sulfate mixture is created with the ratio of alkali metals required for the catalyst application. The mixed sulfate solution can be used directly in the catalyst manufacture process without the need to combine dried individual salts to achieve the desired composition.

In one form of the present invention, the alkali salt solution in the form of hydroxide and carbonate is supported on a ZrO₂/SiO₂ support for the production of a catalyst.

In this form, the catalyst is used for condensation of formaldehyde with alkyl esters or acids to produce ethylenically unsaturated esters or acids. Preferably, methyl propionate is used to produce methyl methacrylate and methacrylate acid.

In accordance with the present invention there is further provided a process of preparing an alkali salt solution when used in the production of catalysts or promoters of catalysts, the process comprising the steps of:
(i) Passing a potassium, rubidium and caesium containing solution to a crystallisation step to obtain a potassium rich salt, and a liquid product comprising rubidium and caesium;
(ii) Further evaporative crystallisation of the liquid product of step (i) to obtain a rubidium rich salt and a liquid product enriched in caesium sulfate; and
(iii) Mixing one or more of the obtained salts and the liquid product at a desired ratio to prepare the alkali salt solution,
   wherein the potassium, rubidium and caesium containing solution is a co-product of a lithium recovery process.

In accordance with the present invention there is still further provided a catalyst produced by any one or more of the processes described hereinabove.

In accordance with the present invention there are yet still further provided promoters of catalysts produced by any one or more of the processes described hereinabove.

### BEST MODE(S) FOR CARRYING OUT THE INVENTION

The present invention provides a process of preparing an alkali salt solution for the production of catalysts or promoters of catalysts, the process comprising the steps of:
(i) Passing a solution containing a mixture of potassium, rubidium and caesium sulfates through an evaporative crystallisation step to obtain a potassium rich salt, and a liquid product or solution, comprising rubidium and caesium sulfate;
(ii) Further evaporative crystallisation of the liquid product of step (i) to obtain a rubidium rich salt and a liquid product or solution, enriched in caesium sulfate; and
(iii) Mixing one or more of the obtained salts and the liquid product at a desired ratio to prepare the alkali salt solution.

The potassium, rubidium and caesium containing solution is, in a preferred form, a co-product of a process for the recovery of lithium from lepidolite. An example of such a process for the recovery of lithium from lepidolite is described in International Patent Application PCT/AU2015/000608 (WO 2016/054683), the entire content of which is incorporated herein by reference.

The potassium containing salt may be potassium sulfate, and the rubidium containing salt may be rubidium sulfate. The rubidium sulfate may be further treated by way of established techniques to yield rubidium carbonate or rubidium hydroxide.

In a preferred form, the caesium containing solution may be caesium sulfate, which can be further treated by way of established techniques to yield caesium carbonate or caesium hydroxide.

It is understood that each of the enriched sulfate solutions produced may be expected to contain minor amounts of each of the other alkali metals contained in the solution of step (i) or alum.

In one form, the alkali salt solution is a promoter in the manufacture of a catalyst. In a further form, the catalyst is a vanadium pentoxide (V₂O₅) catalyst. The vanadium pentoxide catalyst may be used during oxidisation of sulfur dioxide (SO₂) to sulfur trioxide (SO₃).

In one form of the present invention the process provides a potassium sulfate product utilised in fertilisers.

The composition of the potassium, rubidium and caesium containing sulfate can all be represented by the generic formula M₂SO₄, where M represents the alkali metals present and is equal to A+B+C, where A=the molar concentration of potassium, B=the molar concentration of rubidium and C=the molar concentration of caesium.

By mixing portions of the potassium containing salt, the rubidium containing salt and the caesium enriched solution it is possible to create a wide range of compositions for the resulting mixture. Where M=1 the concentration of each of A, B and C can be varied from about 0.1 to 1.0 provided the sum must add to 1.

The alkali sulfate mixture is created with the ratio of alkali metals required for the catalyst application. The mixed sulfate solution can be used directly in the catalyst manufacture process without the need to combine dried individual salts to achieve the desired composition.

In one form of the present invention, the alkali salt solution in the form of hydroxide and carbonate is supported on a ZrO₂/SiO₂ support for the production of a catalyst.

In this form, the catalyst is used for condensation of formaldehyde with alkyl esters or acids to produce ethylenically unsaturated esters or acids. Preferably, methyl propionate is used to produce methyl methacrylate and methacrylate acid.

The present invention further provides a process of preparing an alkali salt solution when used in the production of catalysts or promoters of catalysts, the process comprising the steps of:
(i) Passing a potassium, rubidium and caesium containing solution to a crystallisation step to obtain a potassium rich salt, and a liquid product or solution comprising rubidium and caesium;
(ii) Further evaporative crystallisation of the liquid product of step (i) to obtain a rubidium rich salt and a liquid product or solution enriched in caesium sulfate; and
(iii) Mixing one or more of the obtained salts and the liquid product at a desired ratio to prepare the alkali salt solution, wherein the potassium, rubidium and caesium containing solution is a co-product of a lithium recovery process.

The present invention further provides a catalyst, or a promoter of a catalyst, produced by any one or more of the processes described hereinabove.

The present invention may be better understood with reference to the following non-limiting example.

### Example

In a preferred form of the present invention the solution containing potassium, rubidium and caesium is the co-product of a lithium recovery process described in International Patent Application PCT/AU2015/000608 (WO 2016/054683). The lithium containing material subjected to the described lithium recovery process is lepidolite.

Lepidolite has the chemical formula K(Li,Al)₃(Al,Si)₄O₁₀(F,OH)₂, although it is understood that this may vary, and is a naturally occurring mineral that contain significant quantities of lithium. Further, lepidolites contain a suite of elements with changes in the relative tenors of the elements including potassium, rubidium and caesium. In a typical ore body of lepidolite, the amount of potassium is significantly higher than rubidium and rubidium is higher than of caesium.

A further crystallisation step is utilised to prepare a rubidium containing salt immediately after the crystallisation of the potassium containing salt.

The potassium containing salt, first to be crystallised, is potassium sulfate. The potassium sulfate is removed as a solid and is of such purity that the potassium sulfate product may be utilised to produce potassium sulfate fertilisers.

After potassium sulfate is removed, the rubidium containing salt, being rubidium sulfate, is crystallised and removed, together with any remaining potassium.

The remaining liquid product is rich in both rubidium and caesium.

The products of the crystallisation step (the potassium sulfate, rubidium sulfate and liquid product) are mixed to create a highly concentrated solution with ratios of K:Rb:Cs suitable for use as a promoter in vanadium pentoxide catalysts.

The ratio of the alkali sulfate solution, suitable for use as a promoter in a vanadium pentoxide catalyst is determined by the catalyst manufacture and varies from time to time. The process of the present invention allows the production of an alkali salt mixture in which the ratio of potassium to rubidium to caesium can be varied across a wide range from almost pure potassium sulfate, to a mixture containing significant amounts of each of potassium, rubidium and caesium, to a mixture rich in caesium with lesser amounts of rudibium and low potassium.

The concentration of potassium, rubidium and caesium in the alkali sulfate solution derived from the alum varies with the composition of the lepidolite used in its production. The process described herein is envisaged to be applicable to a wide range of solution compositions as the evaporation and mixing steps enable the desired product composition to be produced regardless of the feed.

The alkali salt solution is used as the promotor in a vanadium pentoxide catalyst. Ratios of the alkali salt solution suitable for addition with V₂O₅ for use as a catalyst is determined by the catalyst manufacturer. It is generally known in the art that the active components of the catalyst are supported on a silica substrate, in a variety of shapes and sizes, optimally designed to allow gas flow through a catalyst bed with a low-pressure differential.

In one form of the present invention, the alkali sulfate solution, after conversion to form hydroxide and carbonate, is used as an addition to a ZrO₂/SiO₂ support for production of a catalyst.

In this form, the catalyst is used for condensation of formaldehyde with alkyl esters or acids to produce ethylenically unsaturated esters or acids. Preferably, methyl propionate is used to produce methyl methacrylate and methacrylate acid.

As can be seen from the above description, the process of the present invention provides a highly concentrated solution with improved ratio of potassium, caesium and rubidium for use in manufacture of a catalyst, without selectively crystallising each of the caesium and rubidium salts.

Modifications and variations such as would be apparent to the skilled addressee are considered to fall within the scope of the present invention.

1-18. (canceled)

19. A process of preparing an alkali salt solution for the production of catalysts or promoters of catalysts, the process comprising the steps of:
(i) passing a solution containing a mixture of potassium, rubidium and caesium sulfates through an evaporative crystallisation step to obtain a potassium containing salt, and a solution comprising rubidium and caesium sulfate;
(ii) further evaporative crystallisation of a liquid product produced by step (i) to obtain a rubidium containing salt and a solution enriched in caesium sulfate; and
(iii) mixing one or more of the obtained salts and the solution of step (ii) at a desired ratio to prepare an alkali salt solution,
wherein the alkali salt solution of step (iii) is used as a catalyst or as a promoter in the manufacture of a catalyst.

20. The process of claim 19, wherein the potassium, rubidium and caesium containing solution of step (i) is a co-product of a process for the recovery of lithium from lepidolite.

21. The process of claim 19, wherein the potassium containing salt is potassium sulfate.

22. The process of claim 19, wherein the rubidium containing salt is rubidium sulfate.

23. The process of claim 22, wherein the rubidium sulfate is further treated to yield rubidium carbonate or rubidium hydroxide.

24. The process of claim 19, wherein one of the solutions of steps (i) or (ii) is further treated to yield caesium carbonate or caesium hydroxide.

25. The process of claim 19, wherein the catalyst is a vanadium pentoxide (V₂O₅) catalyst.

26. The process of claim 25, wherein the vanadium pentoxide catalyst is used in the oxidisation of sulfur dioxide (SO₂) to sulfur trioxide (SO₃).

27. The process of claim 19, wherein the process produces a potassium sulfate product that is in turn utilised in fertilisers.

28. The process of claim 19, wherein the alkali sulfate mixture is created with a ratio of alkali metals determined by an intended catalyst application.

29. The process of claim 19, wherein the composition of the solution containing a mixture of potassium, rubidium and caesium sulfates of step (i) is represented by the generic formula M₂SO₄, where M represents the alkali metals present and is equal to A+B+C, where A equals the molar concentration of potassium, B equals the molar concentration of rubidium, and C equals the molar concentration of caesium.

30. The process of claim 29, wherein the concentration of each of A, B and C is varied from about 0.1 to 0.8 while the sum M equals 1.

31. The process of claim 19, wherein the alkali salt solution of step (iii) is used directly in the catalyst manufacture process without the need to combine dried individual salts to achieve a desired composition.

32. The process of claim 19, wherein the alkali salt solution of step (iii) is supported on a support for the production of the catalyst.

33. The process of claim 19, wherein the alkali salt solution of step (iii) is provided in the form of hydroxide and carbonate, and is supported on a ZrO₂/SiO₂ support for the production of the catalyst.

34. The process of claim 33, wherein the catalyst is used for condensation of formaldehyde with alkyl esters or acids to produce ethylenically unsaturated esters or acids.

35. The process of claim 33, wherein methyl propionate is used to produce methyl methacrylate and methacrylic acid.

36. A process of preparing an alkali salt solution that is used in the production of catalysts or promoters of catalysts, the process comprising the steps of:
(i) passing a potassium, rubidium and caesium containing solution to a crystallisation step to obtain a potassium rich salt, and a solution comprising rubidium and caesium;
(ii) further evaporative crystallisation of a liquid product produced by step (i) to obtain a rubidium rich salt and a solution enriched in caesium sulfate; and
(iii) mixing one or more of the obtained salts and the solutions of steps (i) and (ii) at a desired ratio to prepare an alkali salt solution,
wherein the potassium, rubidium and caesium containing solution of step (i) is a co-product of a lithium recovery process.

37. A catalyst produced by the process set forth in claim 36.

38. A promoter of catalysts produced by the process set forth in claim 36
